(19) United States
(12) Patent Application Publication
(10) Pub. No.: US 2026/0151628 A1
(43) Pub. Date: Jun. 4, 2026

(54) SIGNAL PROCESSING FOR MULTI-DEVICE SYSTEMS

(71) Applicant: Cochlear Limited, Macquarie University, NSW (AU)

(72) Inventor: Jamon WINDEYER, Sydney (AU)

(21) Appl. No.: 19/122,316

(22) PCT Filed: Oct. 4, 2023
(86) PCT No.: PCT/IB2023/059969

§ 371 Date: Apr. 18, 2025

Related U.S. Application Data
(60) Provisional application No. 63/419,151, filed on Oct. 25, 2022.

(51) Int. Cl. A61N 1/36; A61N 1/05; H04R 25/00

(52) U.S. Cl. CPC A61N 1/36038; A61N 1/0541; H04R 25/305; H04R 25/554; H04R 2225/67

(57) ABSTRACT

Presented herein are methods and systems for performing parallel processing of signals at a second medical device when a first medical device is unavailable. The first medical device is configured to deliver treatment to a first portion of a recipient and the first medical device comprises an external component and an implantable component. The second medical device is configured to deliver treatment to a second portion of a recipient. The second medical device is configured to determine that the external component of the first medical device is unavailable and, in response to determining that the external component the first medical device is unavailable, send operating data to the implantable component.

### BACKGROUND

### Field of the Invention

The present invention relates generally to signal processing for multi-device medical device systems, such as binaural hearing systems.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a method is provided. The first method comprises: receiving sound signals at a first hearing device of a recipient; determining, by the first hearing device, that an external component of a second hearing device of the recipient is unavailable; and transmitting, by the first hearing device, operating data associated with the sound signals to an implantable component of the second hearing device in response to determining that the external component is unavailable In another aspect, an implantable medical device system is provided. The implantable medical device system comprises: a first medical device configured to deliver treatment to a first portion of a recipient, wherein, the first medical device comprises an external component and an implantable component; and a second medical configured to deliver treatment to a second portion of a recipient, wherein the second medical device is configured to determine that the external component of the first second hearing device is unavailable and, in response to determining that the external component the first second hearing device of is unavailable, send operating data to the implantable component.

In another aspect, one or more non-transitory computer readable storage media comprising instructions that, when executed by a processor of a first hearing device of a recipient, cause the processor to: receive sound signals; determine that an external component of a second hearing device of the recipient is unavailable; and transmit operating data associated with the sound signals to an implantable component of the second hearing device in response to determining that the external component is unavailable.

In another aspect, medical device is provided. The medical device comprises: one or more input elements configured to receive input signals; memory; one or more processors configured to determining that an external component of a second device is unavailable; and a wireless interface configured to send operating data associated with the input signals to an implantable component of the second device in response to determining that the external component of the second device is unavailable.

### BRIEF DESCRIPTION OF THE DRAWINGS

Embodiments of the present invention are described herein in conjunction with the accompanying drawings, in which:

FIG. 1A is a schematic view of a cochlear implant system in which embodiments presented herein can be implemented;

FIG. 1B is a side view of a recipient wearing the cochlear implant system of FIG. 1A;

FIG. 1C is a schematic view of the components of the cochlear implant system of FIG. 1A;

FIGS. 1D and 1E are block diagrams of sound processing units forming part of the cochlear implant system of FIG. 1A;

FIG. 2A is a schematic view of the components of a bimodal hearing system including cochlear implant and hearing aid in which embodiments presented herein can be implemented.

FIG. 2B is a block diagram of sound processing units forming part of the bimodal hearing system of FIG. 2A.

FIGS. 3A and 3B are block diagrams illustrating an example system in which a hearing aid transmits data to a contralateral cochlear implant, in accordance with certain embodiments presented herein;

FIGS. 4A and 4B are block diagrams illustrating an example system in which a cochlear implant transmits data to a contralateral cochlear implant, in accordance with certain embodiments presented herein;

FIGS. 5A and 5B are block diagrams illustrating another example system in which a cochlear implant transmits data to a contralateral cochlear implant, in accordance with certain embodiments presented herein;

FIGS. 6A and 6B are block diagrams illustrating yet another example system in which a cochlear implant transmits data to a contralateral cochlear implant, in accordance with certain embodiments presented herein;

FIG. 7 is a block diagram illustrating an example of parallel processing of signals, in accordance with certain embodiments presented herein;

FIG. 8 is a flow diagram illustrating an example method of transmitting data to a cochlear implant of a contralateral hearing device when an external component of the contralateral hearing device is unavailable, in accordance with certain embodiments presented herein;

FIG. 9 is a schematic diagram illustrating an example system that can be configured to perform synchronized spectral analysis, in accordance with certain embodiments presented herein; and

FIG. 10 is a schematic diagram illustrating another example system that can be configured to perform synchronized spectral analysis, in accordance with certain embodiments presented herein.

### DETAILED DESCRIPTION

Presented herein are techniques for providing parallel signal processing in a multi-device system. According to embodiments presented herein, a multi-device system is provided that includes at least first and second devices each with separate processing elements. The first device can determine when the processing element of the second device is unavailable and, in response, second operating data to a component of the second device.

Merely for ease of illustration, the techniques presented herein are primarily described with reference to “binaural hearing device systems” or more simply as “binaural systems.” A binaural system includes two hearing devices, where one of the two hearing devices is positioned at each ear of the recipient. More specifically, in a binaural system, each of the two hearing devices operate to convert sound signals into one or more acoustic, mechanical, optical, and/or electrical stimulation signals for delivery to a user/recipient (e.g., each stimulate one of the two ears of the recipient).

The binaural system can include any combination of one or more personal sound amplification products (PSAPs), hearing aids, middle ear auditory prostheses, bone conduction devices, direct acoustic stimulators, tinnitus suppression devices, electro-acoustic prostheses, auditory brain stimulators, cochlear implants, other devices providing acoustic, mechanical, and/or electrical stimulation to a recipient, and/or combinations or variations thereof, etc. For example, embodiments presented herein can be implemented in binaural systems comprising two cochlear implants, a hearing aid and a cochlear implant, different types of cochlear implants, or any other combination of the above or other devices. As such, in certain embodiments, the techniques presented herein enable parallel processing of sound signals by a hearing device of a binaural system when the external component of the contralateral hearing device of the binaural system is unavailable. More specifically, the techniques presented herein enable a first hearing device of the binaural system to transmit signals to an implantable component of a contralateral device of the binaural system when an external component of the contralateral device is unavailable.

As noted, reference to binaural systems is merely illustrative and it is to be appreciated that the techniques presented herein can be implemented in other types of multi-device systems. For example, the techniques presented herein can be implemented with any of a number of systems, including in conjunction with cochlear implants or other hearing devices, balance prostheses (e.g., vestibular implants), retinal or other visual prostheses, cardiac devices (e.g., implantable pacemakers, defibrillators, etc.), seizure devices, sleep apnea devices, electroporation devices, spinal cord stimulators, deep brain stimulators, motor cortex stimulators, sacral nerve stimulators, pudendal nerve stimulators, vagus/vagal nerve stimulators, trigeminal nerve stimulators, diaphragm (phrenic) pacers, pain relief stimulators, other neural, neuromuscular, or functional stimulators, etc. In further embodiments, the presented herein can also be implemented by, or used in conjunction with, systems comprising remote microphone devices, consumer electronic devices, etc.

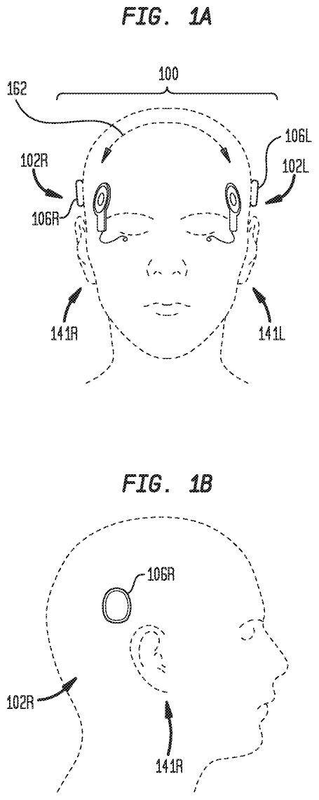

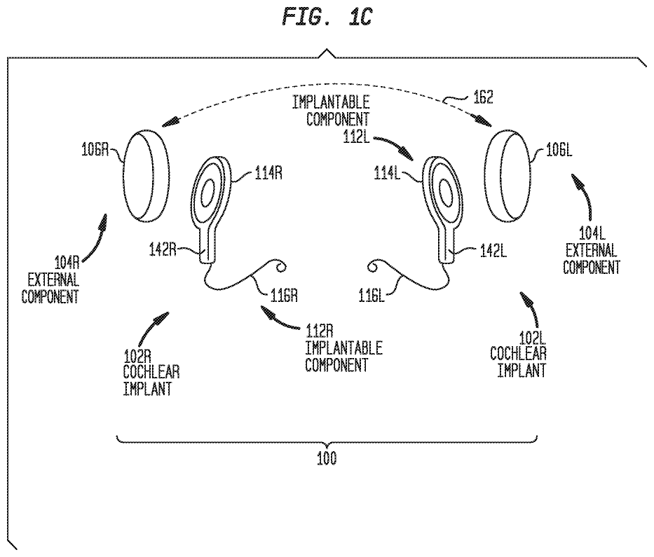

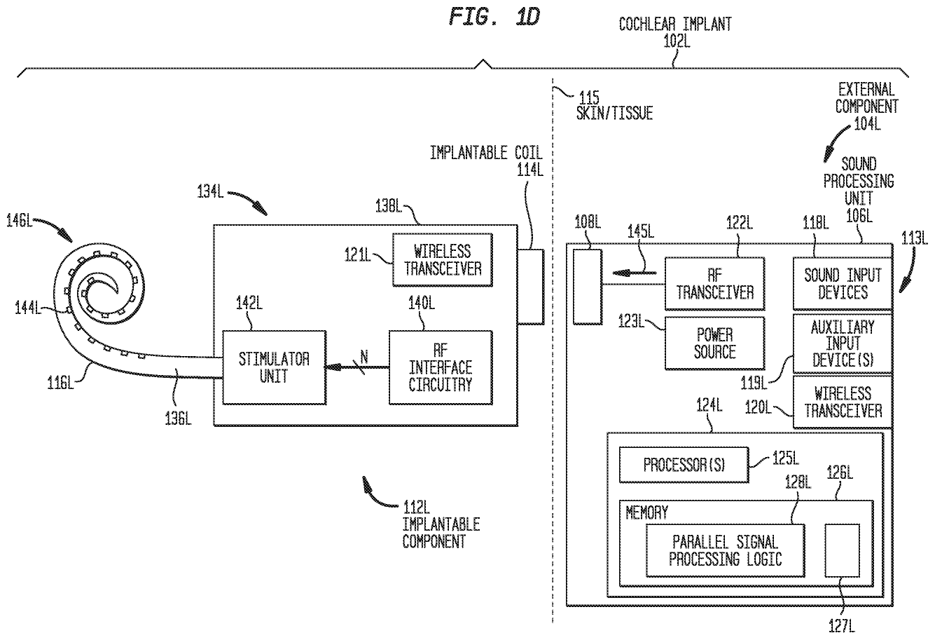

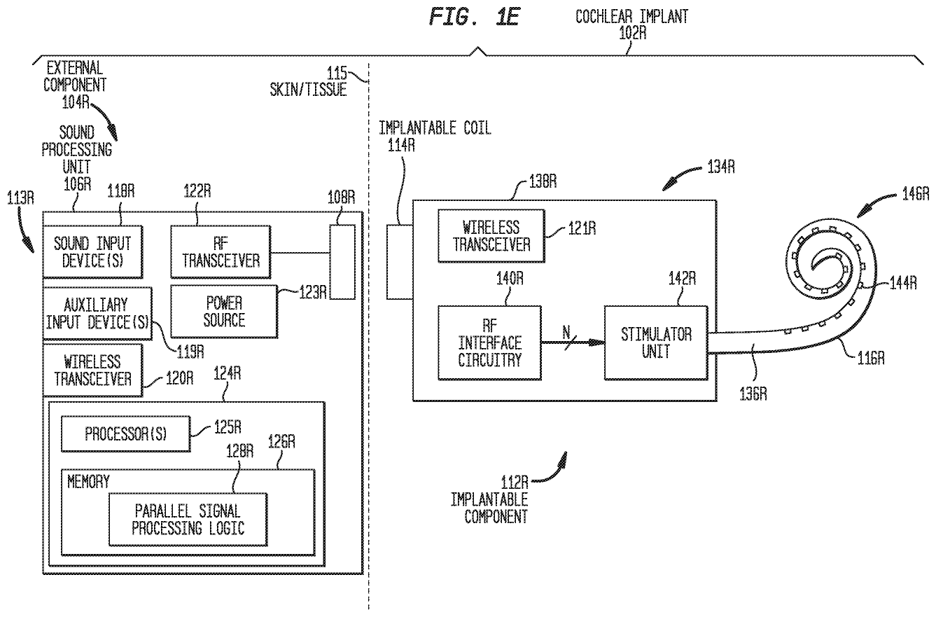

FIGS. 1A-1E are diagrams illustrating one example bilateral cochlear implant system 100 configured to implement the techniques presented herein. As used herein, a “bilateral cochlear implant system” is a specific type of binaural system that includes first and second cochlear implants located at first and second ears, respectively, of a recipient. In such systems, each of the two cochlear implant systems delivers stimulation (current) pulses to one of the two ears of the recipient (i.e., either the right or the left ear of the recipient). In a bilateral cochlear implant system, one or more of the two cochlear implants can also deliver acoustic stimulation to the ears of the recipient (e.g., an electro-acoustic cochlear implant) and/or the two cochlear implants need not be identical with respect to, for example, the number of electrodes used to electrically stimulate the cochlea, the type of stimulation delivered, a type of the cochlear implant (e.g., whether the cochlear implant includes an external component or is totally implantable), etc.

More specifically, FIGS. 1A-1E illustrate an example bilateral system 100 comprising left and right cochlear implants, referred to as cochlear implant 102L and cochlear implant 102R. FIGS. 1A and 1B are schematic drawings of a recipient wearing the left cochlear implant 102L at a left ear 141L and the right cochlear implant 102R at a right ear 141R, while FIG. 1C is a schematic view of each of the left and right cochlear implants. FIGS. 1D and 1E are block diagrams illustrating further details of the left cochlear implant 102L and the right cochlear implant 102R, respectively.

Referring specifically to FIG. 1C, cochlear implant 102L includes an external component 104L that is configured to be directly or indirectly attached to the body of the recipient and an implantable component 112L configured to be implanted in the recipient. The external component 104L comprises a sound processing unit 106L, while the implantable component 112L includes an internal coil 114L, a stimulator unit 142L and an elongate stimulating assembly (electrode array) 116L implanted in the recipient's left cochlea (not shown in FIG. 1C).

The cochlear implant 102R is substantially similar to cochlear implant 102L. In particular, cochlear implant 102R includes an external component 104R comprising a sound processing unit 106R, and an implantable component 112R comprising internal coil 114R, stimulator unit 142R, and elongate stimulating assembly 116R.

As noted above, the cochlear implant 102R includes the sound processing unit 106R and the implantable component 112R and cochlear implant 102L includes the sound processing unit 106L and the implantable component 112L. However, with some hearing devices (e.g., a totally implantable cochlear implant (TICI)), the cochlear implant captures sound signals itself via implantable sound sensors and then uses those sound signals as the basis for delivering stimulation signals to the recipient.

FIG. 1D is a block diagram illustrating further details of cochlear implant 102L, while FIG. 1E is a block diagram illustrating further details of cochlear implant 102R. As noted, cochlear implant 102R is substantially similar to cochlear implant 102L and includes like elements as that described below with reference to cochlear implant 102L. For ease of description, further details of cochlear implant 102R have been omitted from the description.

As noted, the external component 104L of cochlear implant 102L includes a sound processing unit 106L. The sound processing unit 106L comprises one or more input devices 113L that are configured to receive input signals (e.g., sound or data signals). In the example of FIG. 1D, the one or more input devices 113L include one or more sound input devices 118L (e.g., microphones, audio input ports, telecoils, etc.), one or more auxiliary input devices 119L (e.g., audio ports, such as a Direct Audio Input (DAI), data ports, such as a Universal Serial Bus (USB) port, cable port, etc.), and a wireless transmitter/receiver (transceiver) 120L. However, it is to be appreciated that one or more input devices 113L can include additional types of input devices and/or less input devices (e.g., one or more auxiliary input devices 119L could be omitted).

The sound processing unit 106L also comprises one type of a closely-coupled transmitter/receiver (transceiver) 122L, referred to as or radio-frequency (RF) transceiver 122L, a power source 123L, and a processing module 124L. The processing module 124L comprises one or more processors 125L and a memory 126L that includes sound processing logic 127L and parallel signal processing logic 128L. Parallel sound processing logic 128L can be configured to process signals for transmission to implantable component 112L and implantable component 112R in a situation in which sound processing unit 106R is unavailable. Parallel sound processing logic 128L can process signals for transmission to implantable component 112L and signals for transmission to implantable component 112R in different ways based on a number of different factors.

In the examples of FIGS. 1A-1E, the sound processing unit 106L and the sound processing unit 106R are off-the-ear (OTE) sound processing units (i.e., components having a generally cylindrical shape and which is configured to be magnetically coupled to the recipient's head), etc. However, it is to be appreciated that embodiments of the present invention can be implemented by sound processing units having other arrangements, such as by a behind-the-ear (BTE) sound processing unit configured to be attached to and worn adjacent to the recipient's ear, including a mini or micro-BTE unit, an in-the-canal unit that is configured to be located in the recipient's ear canal, a body-worn sound processing unit, etc.

The implantable component 112L comprises an implant body (main module) 134L, a lead region 136L, and the intra-cochlear stimulating assembly 116L, all configured to be implanted under the skin/tissue (tissue) 115 of the recipient. The implant body 134L generally comprises a hermetically-sealed housing 138L in which RF interface circuitry 140L, a wireless transceiver 121L, and a stimulator unit 142L are disposed. The implant body 134L also includes the internal/implantable coil 114L that is generally external to the housing 138L, but which is connected to the transceiver 140L via a hermetic feedthrough (not shown in FIG. 1D).

As noted, stimulating assembly 116L is configured to be at least partially implanted in the recipient's cochlea. Stimulating assembly 116L includes a plurality of longitudinally spaced intra-cochlear electrical stimulating contacts (electrodes) 144L that collectively form a contact or electrode array 146L for delivery of electrical stimulation (current) to the recipient's cochlea.

Stimulating assembly 116L extends through an opening in the recipient's cochlea (e.g., cochleostomy, the round window, etc.) and has a proximal end connected to stimulator unit 142L via lead region 136L and a hermetic feedthrough (not shown in FIG. 1D). Lead region 136L includes a plurality of conductors (wires) that electrically couple the electrodes 144L to the stimulator unit 142L.

As noted, the cochlear implant 102L includes the external coil 108L and the implantable coil 114L. The coils 108L and 114L are typically wire antenna coils each comprised of multiple turns of electrically insulated single-strand or multi-strand platinum or gold wire. Generally, a magnet is fixed relative to each of the external coil 108L and the implantable coil 114L. The magnets fixed relative to the external coil 108L and the implantable coil 114L facilitate the operational alignment of the external coil 108L with the implantable coil 114L. This operational alignment of the coils enables the external component 104L to transmit data, as well as possibly power, to the implantable component 112L via a closely-coupled wireless link formed between the external coil 108L with the implantable coil 114L. In certain examples, the closely-coupled wireless link is a radio frequency (RF) link. However, various other types of energy transfer, such as infrared (IR), electromagnetic, capacitive and inductive transfer, can be used to transfer the power and/or data from an external component to an implantable component and, as such, FIG. 1D illustrates only one example arrangement.

As noted above, sound processing unit 106L includes the processing module 124L. The processing module 124L is configured to convert received input signals (received at one or more of the input devices 113L) into output signals 145L for use in stimulating a first ear of a recipient (i.e., the processing module 124L is configured to perform sound processing on input signals received at the sound processing unit 106L). Stated differently, in the sound processing mode, the one or more processors 125L are configured to execute sound processing logic stored, for example, in in memory 126L to convert the received input signals into output signals 145L that represent electrical stimulation for delivery to the recipient.

In the embodiment of FIG. 1D, the output signals 145L are provided to the RF transceiver 122L, which transcutaneously transfers the output signals 145L (e.g., in an encoded manner) to the implantable component 112L via external coil 108L and implantable coil 114L. That is, the output signals 145L are received at the RF interface circuitry 140L via implantable coil 114L and provided to the stimulator unit 142L. The stimulator unit 142L is configured to utilize the output signals 145L to generate electrical stimulation signals (e.g., current signals) for delivery to the recipient's cochlea via one or more stimulating contacts 144L. In this way, cochlear implant 102L electrically stimulates the recipient's auditory nerve cells, bypassing absent or defective hair cells that normally transduce acoustic vibrations into neural activity, in a manner that causes the recipient to perceive one or more components of the received sound signals.

As noted, cochlear implant 102R is substantially similar to cochlear implant 102L and comprises external component 104R and implantable component 112R. External component 104R includes a sound processing unit 106R that comprises external coil 108R, input devices 113R (i.e., one or more sound input devices 118R, one or more auxiliary input devices 119R, and wireless transceiver 120R), closely-coupled transceiver (RF transceiver) 122R, power source 123R, and processing module 124R. The processing module 124R includes one or more processors 125R and a memory 126R that includes sound processing logic 127R and parallel signal processing logic 128R. The implantable component 112R includes an implant body (main module) 134R, a lead region 136R, and the intra-cochlear stimulating assembly 116R, all configured to be implanted under the skin/tissue (tissue) 115 of the recipient. The implant body 134R generally comprises a hermetically-sealed housing 138R in which RF interface circuitry 140R, a wireless transceiver 121R, and a stimulator unit 142R are disposed. The implant body 134R also includes the internal/implantable coil 114R that is generally external to the housing 138R, but which is connected to the RF interface circuitry 140R via a hermetic feedthrough (not shown in FIG. 1E). The stimulating assembly 116R includes a plurality of longitudinally spaced intra-cochlear electrical stimulating contacts (electrodes) 144R that collectively form a contact or electrode array 146R for delivery of electrical stimulation (current) to the recipient's cochlea. Each of the elements of cochlear implant 102R shown in FIG. 1E are similar to like-numbered elements of cochlear implant 102L shown in FIG. 1D.

It is to be appreciated that the arrangements of cochlear implants 102L and 102R, as shown in FIGS. 1A-1E, are merely illustrative and that the cochlear implants 102L and 102R could have different arrangements. For example, in certain embodiments, the implantable components 112L and 112R could each include a wireless transceiver that is similar to the wireless transceivers 120L and 120R. In the same or other embodiments, the implantable components 112L and 112R could each include processing modules that are similar to the processing modules 124L and 124R. The implantable components 112L and 112R could also include processing modules that are not necessarily the same as the processing modules 124L and 124R, for example, in terms of functional capabilities.

The cochlear implants 102L and 102R are configured to establish one or more binaural wireless communication link/channels 162 (binaural wireless link) that enables the cochlear implants 102L and 102R (e.g., the sound processing units 104L/104R and/or the implantable components 1121/122, if equipped with wireless transceivers) to wirelessly communicate with one another. The binaural wireless link(s) 162 can be, for example, magnetic induction (MI) links, standardized wireless channel(s), such as a Bluetooth®, Bluetooth® Low Energy (BLE) or other channel interface making use of any number of standard wireless streaming protocols, wireless channel(s) using proprietary protocols for wireless exchange of data, etc. Bluetooth ® is a registered trademark owned by the Bluetooth® SIG. The binaural wireless link(s) 162 is/are enabled by the wireless transceivers 120L and 120R.

The sound processing performed at each of the cochlear implant 102L and the cochlear implant 102R (e.g., at the sound processing units 104L/104R and/or the implantable components 112L/112R, if equipped with processing modules) includes some form of parallel processing (e.g., some means to process received sound signals in a parallel fashion for output to a recipient and to a contralateral hearing device).

For a binaural hearing device system, such as bilateral cochlear implant system 100, parallel processing of sound signals is important in a situation in which a sound processing unit 106L/106R of a cochlear implant 102L/102R is unavailable. For example, if sound processing unit 106L is unavailable, sound processing unit 106R can process sound signals in parallel and in different ways. In this example, sound processing unit 106R can output the processed sound signals (e.g., that were processed in a first way) to a recipient of bilateral cochlear implant system 100 and can transmit the processed sound signals (e.g., that were processed in a different way) to implantable component 112L for output to the recipient. In addition, sound processing unit 106R can output different types of data to the recipient and to implantable component 112L.

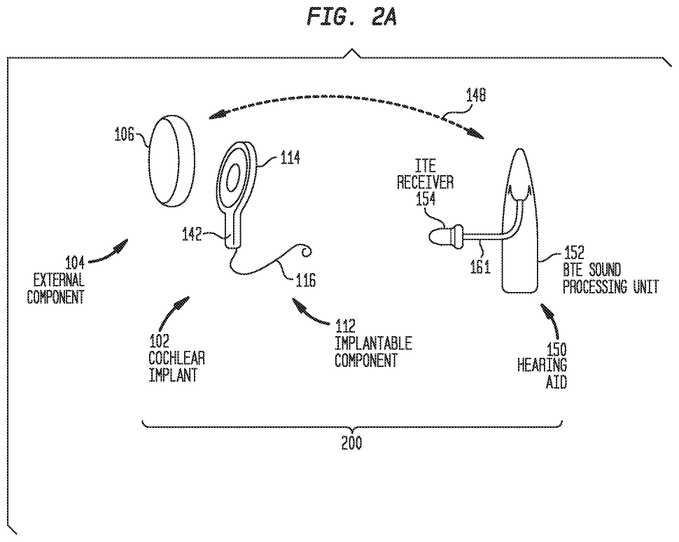

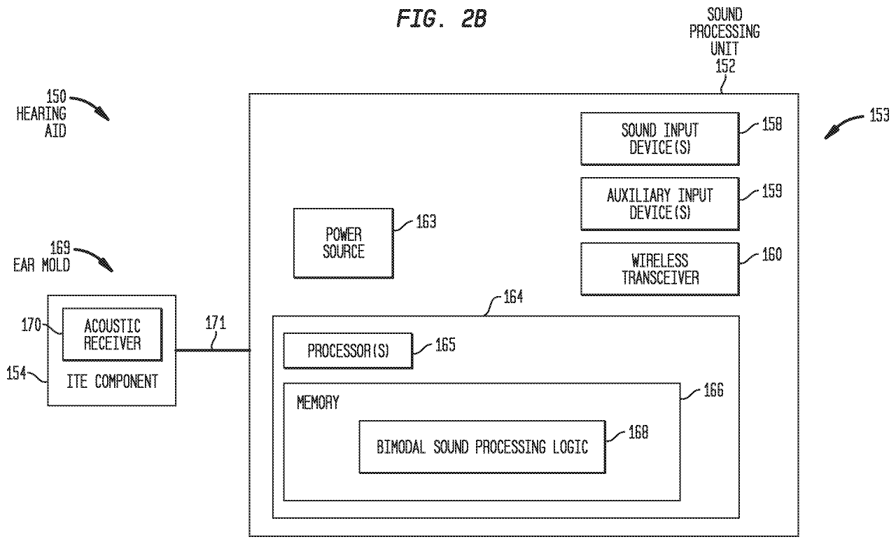

FIGS. 2A and 2B are diagrams illustrating another example binaural system 200 configured to implement the techniques presented herein. More specifically, FIG. 2A illustrates an example binaural system 200 comprising a cochlear implant, referred to as cochlear implant 102, and a hearing aid 150, each shown separate from the head of the recipient. FIG. 2B is a block diagram illustrating further details of hearing aid 150.

The cochlear implant 102 is substantially similar to cochlear implants 102L and 102R. In particular, cochlear implant 102 includes an external component 104 that includes a sound processing unit 106 and an implantable component 112 comprising internal coil 114, stimulator unit 142, and elongate stimulating assembly 116. As shown in FIG. 2A, hearing aid 150 comprises a sound processing unit 152 and an in-the-ear (ITE) component 154.

In the embodiment of FIG. 2A, the hearing aid 150 (e.g., sound processing unit 152) and the cochlear implant 102 (e.g., sound processing unit 106) communicate with one another over a wired or wireless communication channel/link 148. The communication channel 148 is a bidirectional communication channel and can be, for example, a magnetic inductive (MI) link, a short-range wireless link, such as a Bluetooth® link that communicates using short-wavelength Ultra High Frequency (UHF) radio waves in the industrial, scientific and medical (ISM) band from 2.4 to 2.485 gigahertz (GHz), or another type of wireless link. Bluetooth® is a registered trademark owned by the Bluetooth ® SIG.

As illustrated in FIG. 2B, hearing aid 150 comprises a sound processing unit 152 and an in-the-ear (ITE) component 154. The sound processing unit 152 comprises one or more input devices 153 that are configured to receive input signals (e.g., sound or data signals). In the example of FIG. 2B, the one or more input devices 153 include one or more sound input devices 158 (e.g., microphones, audio input ports, telecoils, etc.), one or more auxiliary input devices 159 (e.g., audio ports, such as a Direct Audio Input (DAI), data ports, such as a Universal Serial Bus (USB) port, cable port, etc.), and a wireless transmitter/receiver (transceiver) 160. However, it is to be appreciated that one or more input devices 153 can include additional types of input devices and/or less input devices (e.g., the wireless transceiver 160 and/or one or more auxiliary input devices 159 could be omitted).

The sound processing unit 152 also comprises a power source 163, and a processing module 164. The processing module 164 comprises one or more processors 165 and a memory 166 that includes bimodal sound processing logic 168. The bimodal sound processing logic 168 can be configured to communicate with cochlear implant 102 (e.g., via link 148) and to process signals for transmission to implantable component 112 when sound processing unit 106 is unavailable.

As noted, the hearing aid 150 also comprises an ITE component 154. The ITE component 154 comprises an ear mold 169 and an acoustic receiver 170 disposed in the ear mold. The ear mold 169 is configured to positioned/inserted into the ear canal of the recipient and retained therein. The acoustic receiver 170 is electrically connected to the sound processing unit 152 via a cable 171.

As noted above, sound processing unit 152 includes the processing module 164. The processing module 164 is configured to convert received input signals (received at one or more of the one or more input devices 153) into output signals for use in stimulating an ear of a recipient (i.e., the processing module 164 is configured to perform sound processing on input signals received at the sound processing unit 152). Stated differently, the one or more processors 165 are configured to execute bimodal sound processing logic 168 in memory 166 to convert the received input signals into processed signals that represent acoustic stimulation for delivery to the recipient.

In the embodiment of FIG. 2B, the processed signals are provided to the acoustic receiver 170 (via cable 171), which in turn acoustically stimulates the ear of the recipient. That is, the processed signals, when delivered to the acoustic receiver 170, cause the acoustic receiver to deliver acoustic stimulation signals (acoustic output signals) to the ear of the recipient. The acoustic stimulation signals cause vibration of the ear drum that, in turn, induces motion of the cochlea fluid causing the recipient to perceive the input signals received at the one or more of the input devices 153.

FIG. 2B illustrates one specific example arrangement for hearing aid 150. However, it is to be appreciated that embodiments of the present invention can be implemented with hearing aids having alternative arrangements.

In a binaural system (e.g., system 100, system 200, etc.) presented herein, various components at each side of the head can communicate with one another. For example, in the example of FIGS. 1A-1E, external component 104R can communicate with both implantable component 112L (i.e., the ipsilateral implant) and external component 104L (i.e., the contralateral external component) via medium/long range data links such as MI or 2.4 GHz. However, conventional systems do not include such inter-connectivity and each implantable component of a hearing device is still dependent on the ipsilateral sound processor for delivering audio/electrical stimulation data to use as output. Therefore, if a sound processor is unavailable, the recipient would be unable to use the entire ipsilateral hearing device. In this situation, the recipient can be forced to use a single hearing device instead of the two hearing devices.

A sound processor can be unavailable for a number of reasons. For example, the battery can be depleted or the sound processor can be charging, can be misplaced, be undergoing repair, purposely turned off to conserve battery, etc. According to embodiments presented herein, a hearing device can detect when a sound processor in the contralateral hearing device is unavailable and transmit operating data to the implantable portion of the contralateral hearing device to maintain binaural sound processing for the recipient. The operating data can include, for example, electrical stimulation data or processed (e.g., channelized, compressed, etc.) audio signals. According to some embodiments, when a single hearing device is sending signal information to two implants, the signal processing for each implant can be different. In these cases, adjustments can be made to either the ‘front end’ signal processing (e.g., directional processing, noise reduction, gain adjustments, etc.) or the ‘back end’ signal processing. By transmitting data to a contralateral implant when the contralateral sound processor is unavailable, a recipient can maintain binaural sound processing when only one hearing device is available.

In certain aspects, embodiments described herein provide for entering a “single sided mode” in which a first hearing device initiates a connection with and sends data to an implantable component of a contralateral second hearing device based on determining that a processing unit of the contralateral second hearing device is unavailable. Embodiments described herein further provide for adjusting the signal processing for the contralateral implantable component without changing the signal processing for the ipsilateral side.

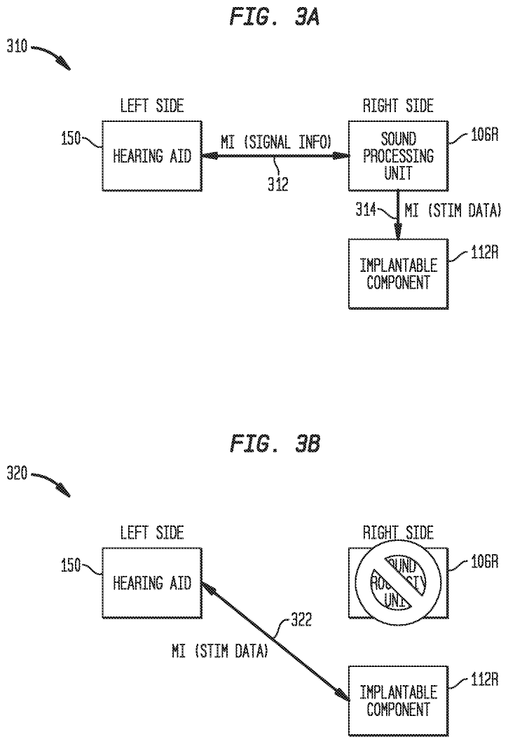

FIGS. 3A and 3B are diagrams illustrating an example binaural hearing system (binaural system) comprising a hearing aid and a cochlear implant that is configured to implement the techniques presented herein.

The binaural system 310 illustrated in FIG. 3A includes a hearing aid 150 on the left side and a cochlear implant on the right that includes a sound processing unit 106R and an implantable component 112R. Although system 310 illustrates the hearing aid 150 on the left side and the cochlear implant on the right side, the configuration is exemplary and the hearing aid could be on the right side while the cochlear implant is on the left side.

As illustrated in FIG. 3A, hearing aid 150 and sound processing unit 106R exchange information, such as signal information for binaural sound processing, across link 312. In this example, link 312 is an MI link. Sound processing unit 106R and implantable component 112R additionally exchange data, such as stimulation data, unprocessed audio data, at least partially processed audio data, etc. over link 314. In this example, link 314 is an MI link.

FIG. 3B illustrates an example system 320, in which sound processing unit 106R is unavailable. For example, the battery of sound processing unit 106R can be depleted, the battery can be charging, a user can have turned off sound processing unit 106R, or sound processing unit 106R can be unavailable for another reason. In the example illustrated in FIG. 3B, hearing aid 150 detects that sound processing unit 106R is unavailable. For example, hearing aid 150 can detect that sound processing unit 106R is unavailable by detecting that link 312 with sound processing unit 106R is unavailable. As another example, implantable component 112R can detect that sound processing unit 106R is unavailable and can transmit a message to hearing aid 150 indicating that sound processing unit 106R is unavailable.

When hearing aid 150 detects that sound processing unit 106R is unavailable, hearing aid 150 can enter “single sided mode.” In one embodiment, hearing aid 150 can prompt the recipient of the hearing aid 150 to enter single sided mode (e.g., via an external device) and the recipient can select an option to enter single sided mode. In another embodiment, hearing aid 150 can automatically enter single sided mode based on detecting that sound processing unit 106R is unavailable.

When hearing aid 150 enters single sided mode, hearing aid 150 forms a link 322 with implantable component 112R to send data (such as stimulation data, unprocessed audio data, at least partially processed audio data, etc.) to implantable component 112R. For example, hearing aid 150 can process sound input (such as from sound input device(s) 158) to form data, such as stimulation data or audio data. Hearing aid 150 can additionally use the previously established MI link (e.g., link 312) to form link 322 for sending the data to implantable component 112R. In some embodiments, the type of data transmitted to implantable component 112R can be based on a type of the link (e.g., MI, 2.4 GHz, etc.) established between hearing aid 150 and link 322. Because hearing aid 150 sends the stimulation or audio data to implantable component 112R, the recipient receives acoustic output from hearing aid 150 and simultaneously receives electrical stimulation from implantable component 112R. Therefore, the recipient remains “on air” on both sides even when sound processing unit 106R is unavailable.

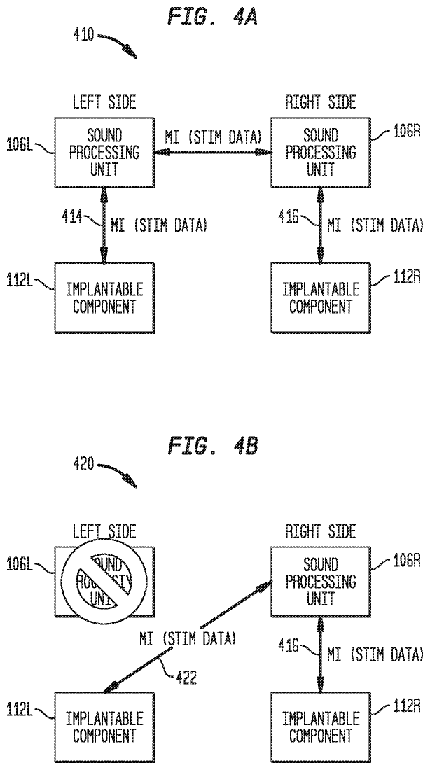

FIGS. 4A and 4B are diagrams illustrating an example binaural system that comprises two cochlear implants and is configured to implement the techniques presented herein. FIG. 4A illustrates a system 410 in which the left side and the right side both include cochlear implants of the same type (e.g., external components and implantable components). More specifically, the left side includes sound processing unit 106L and implantable component 112L and the right side includes sound processing unit 106R and implantable component 112R. In a normal operating mode, sound processing units 106L and 106R communicate over link 412 to exchange signal information. In addition, sound processing unit 106L communicates with implantable component 112L via link 414 and sound processing unit 106R communicates with implantable component 112R via link 416 to exchange data, such as stimulation data. In the example illustrated in FIG. 4A, links 412, 414, and 416 are MI links.

FIG. 4B illustrates an example system 420 in which sound processing unit 106L becomes unavailable. Although in this example, sound processing unit 106L becomes unavailable, in other examples sound processing unit 106R can become unavailable and sound processing unit 106L can perform the functions described below.

Sound processing unit 106R can detect that sound processing unit 106L is unavailable (e.g., by detecting that link 412 is unavailable or by receiving a message from implantable component 112L) and sound processing unit 106R can enter “single sided mode.” As described above with respect to FIGS. 3A and 3B, sound processing unit 106R can enter single sided mode automatically or in response to a selection by the recipient of the binaural system. When sound processing unit 106R enters single sided mode, sound processing unit 106R can form link 422 with implantable component 112L (e.g., a MI link) and can transmit data to implantable component 112L via link 422. For example, sound processing unit 106R can process input data received from input devices (such as sound input devices 118L) to form data, such as stimulation data, unprocessed audio data, or at least partially processed audio data, to transmit to implantable component 112L via link 422.

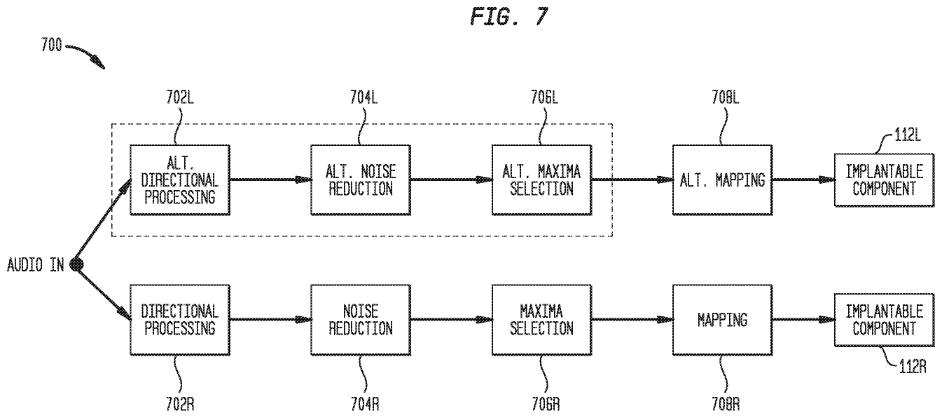

In one embodiment, sound processing unit 106R can send stimulation data to implantable component 112L while simultaneously sending stimulation data to implantable component 112R. As described below with respect to FIG. 7, sound processing unit 106R can adjust processing of the data received from the input devices to create the stimulation data transmitted to implantable component 112L without adjusting processing of the input data to create the stimulation data transmitted to implantable component 112R. In another embodiment, sound processing unit 106R can send unprocessed audio data or at least partially processed audio data to implantable component 112L and implantable component 112L can process the sound data to produce stimulation data. The at least partially processed audio data can take a number of different forms and be a result of any of a number of different processing operations. For example, the at least partially processed audio data can comprise compressed audio signals/data, channelized signals, etc. In another embodiment, sound processing unit 106R can send the audio data (unprocessed or at least partially processed) to implantable component 112L while simultaneously sending the stimulation data to implantable component 112R.

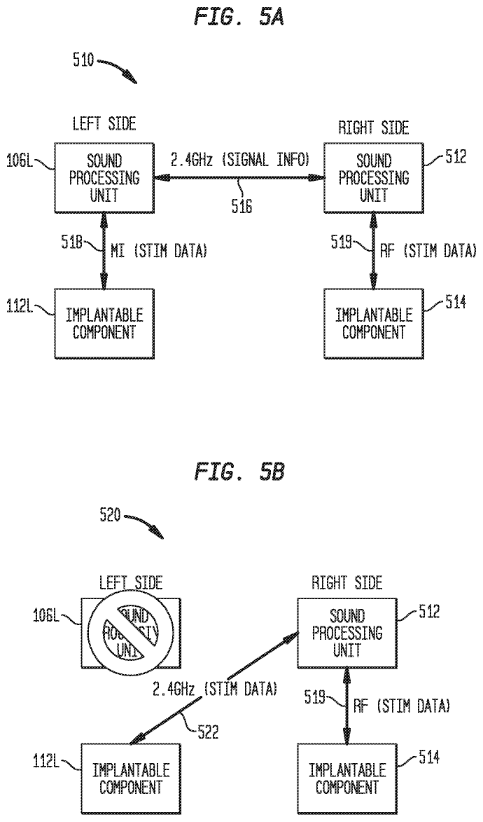

FIGS. 5A and 5B are diagrams illustrating an example in which a binaural system that comprises two different types of cochlear implants configured to implement the techniques presented herein.

FIG. 5A illustrates a system 510 in which the left side includes a cochlear implant with a sound processing unit 106L and an implantable component 112 and the right side includes a cochlear implant sound processor 512 and an implantable component 514. In other implementations, the components can be on different sides. In the normal operating mode, implantable component 112L receives stimulation data from sound processing unit 106L via link 518 (e.g., an MI link) and the implantable component 514 receives stimulation data from the sound processing unit 512 via link 519 (e.g., a radio frequency (RF) link). Sound processing unit 106L and sound processing unit 512 can additionally share signal information via link 516 (e.g., a 2.4 GHz data link).

FIG. 5B illustrates a system 520 in which sound processing unit 106L is unavailable. sound processing unit 512 can detect that sound processing unit 106L is unavailable (e.g., when link 516 is unavailable or based on receiving a message from implantable component 112L) and sound processing unit 512 can enter “single sided mode.” As described above, sound processing unit 512 can enter single sided mode automatically or in response to a selection by the recipient of the binaural system. When sound processing unit 512 enters single sided mode, sound processing unit 512 can form link 522 with implantable component 112L (e.g., a 2.4 GHz link) and can transmit data to implantable component 112L via link 522. In this example, link 522 is the same type of link as link 516 (e.g., a 2.4 GHz link). Sound processing unit 512 can receive input signals (e.g., sound input signals received at a sound input device, such as a microphone) and process the input signals to produce the data transmitted to implantable component 112L via link 522.

In one embodiment, sound processing unit 512 can send stimulation data to implantable component 514 via RF link 519 while simultaneously sending stimulation data to implantable component 112R via the 2.4 GHz link 522. As described below with respect to FIG. 7, sound processing unit 512 can adjust processing of the input signals to produce the data transmitted to implantable component 112L without adjusting processing of the input signals to produce the data transmitted to implantable component 514. In another embodiment, sound processing unit 512 can send processed sound data (e.g., compressed sound input data) to implantable component 112L and implantable component 112L can process the sound data to produce stimulation data. In another embodiment, sound processing unit 512 can send audio data (unprocessed or at least partially processed) to implantable component 112L via the 2.4 GHz link 522 while simultaneously sending stimulation data to implantable component 514 via RF link 519.

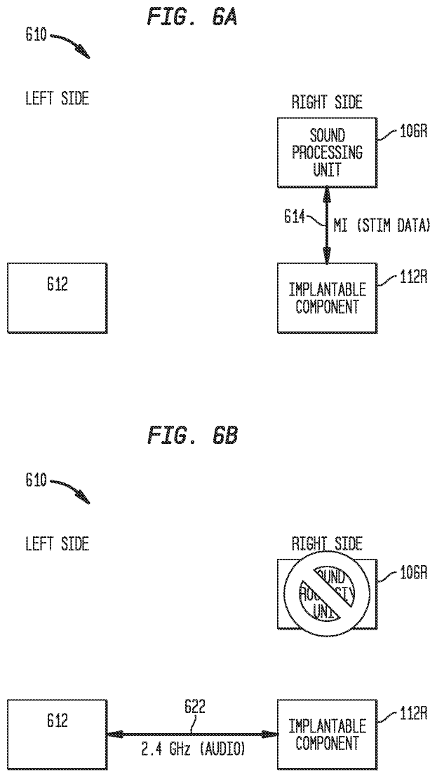

FIGS. 6A and 6B are diagrams illustrating an example in which a binaural system that comprises a totally implantable cochlear implant and a cochlear implant with an external component, configured to implement the techniques presented herein. More specifically, FIG. 6A illustrates a system 610 in which the left side includes a totally implantable cochlear implant 612 and the right side includes a cochlear implant including sound processing unit 106R and implantable component 112R. Although the totally implantable cochlear implant 612 is shown on the left and the sound processing unit 106R/implantable component 112R is shown on the right in FIG. 6A, the positions can be reversed. In a normal operating mode, totally implantable cochlear implant 612 obtains stimulation data through processing the internal microphone. Additionally, sound processing unit 106R communicates with implantable component 112R via link 614 (e.g., an MI link).

FIG. 6B illustrates an example system 620 in which sound processing unit 106R becomes unavailable. In this example, implantable component 112R can detect that sound processing unit 106R is unavailable and implantable component 112R can initiate a connection with totally implantable cochlear implant 612 via link 622 (e.g., a 2.4 GHz link). In response to receiving the indication that sound processing unit 106R is unavailable, totally implantable cochlear implant 612 can enter single sided mode. In this example, totally implantable cochlear implant 612 can transmit data, such as compressed microphone samples from the internal microphone of totally implantable cochlear implant 612, to implantable component 112R via link 622. The type of data transmitted to implantable component 112R can be based on a type of link established between the two hearing devices. Implantable component 112R can process the compressed microphone samples to produce stimulation to deliver to the recipient.

Even though sound processing unit 106R is unavailable, implantable component 112R is still able to process sound signals received from totally implantable cochlear implant 612 to produce the stimulation data for the right ear. Therefore, the recipient is able to receive binaural stimulation data when the sound processing unit 106R is unavailable.

FIG. 7 is a diagram illustrating parallel processing of signals by a hearing device when a sound processing unit of a contralateral hearing device in a binaural system is unavailable.

FIG. 7 illustrates a system 700 in which a sound processing unit 106L of a cochlear implant (e.g., cochlear implant 102L) is unavailable and a contralateral sound processing unit, such as sound processing unit 106R of cochlear implant 102R, is performing parallel processing of signals for cochlear implant 102R and cochlear implant 102L. In some situations, the parallel processing can be performed by hearing aid 150, sound processing unit 512, or another device other than cochlear implant 102R.

Cochlear implant 102R can receive audio, such as from sound input device(s) 118R of cochlear implant 102R and, at 702R, sound processing unit 106R can perform directional processing of the sound signal (e.g., for output to the ipsilateral implantable component 112R). At 704R, sound processing unit 106R can perform noise reduction processing of the signal and, at 706R, sound processing unit 106R can perform maxima selection or a different channel selection method.

Concurrently, at 702L, sound processing unit 106R can additionally perform alternate sound processing of the sound signal for output of a stimulation signal to the contralateral implantable component 112L. For example, sound processing unit 106R can process the sound signals received at sound input device(s) 118R in a different or alternate manner for transmission to contralateral implantable component 112L. At 702L, sound processing unit 106R can perform alternate noise reduction processing of the alternate signal and, at 706L, sound processing unit can perform alternate maxima selection. Because the stimulation data associated with the sound signal is to be transmitted to both a right ear and a left ear of the recipient (and possibly to a different type of hearing device), the processing at each step (e.g., sound processing, noise reduction, maxima selection) can be different for a signal that is to be transmitted to an ipsilateral hearing device and a signal that is to be transmitted to a contralateral hearing device.

Additionally, the processing at 702L, 704L, and 706L is optional. For example, sound processing unit 106R can perform directional processing 702R, noise reduction processing 704R, and maxima selection 706R on the sound signal to produce the stimulation signal that is to be transmitted to the ipsilateral implantable component 112R without performing the alternate steps on the signal that is to be transmitted to contralateral implantable component 112L. In some embodiments, implantable component 112L can perform processing on the signal after receiving the signal from sound processing unit 106R.

At 708R, sound processing unit 106R performs mapping of the signal and transmits the stimulation data to implantable component 112R. At 708L, sound processing unit 106L performs alternate mapping of the alternate signal and transmits the signal to implantable component 112L.

When performing the alternate signal processing steps, adjustments can be made to either the ‘front end’ signal processing (e.g., directional processing, noise reduction, gain adjustments, etc.) or the ‘back-end’ signal processing. These adjustments could be made in various ways and for various reasons. For example, any aspect of processing (front or back end) could be adjusted to match the processing more closely for the contralateral side in a normal configuration (i.e., match the map parameters on the contralateral signal processor). These parameters could be sent from the contralateral implantable component when the single sided mode is entered or otherwise stored in the system/device for use when required.

In some embodiments, certain back-end adjustments can be made to match the requirements imposed by characteristics of the contralateral implant hardware (e.g., number of available electrodes, stimulation rate limits, etc.) and physiology. Such adjustments could include adjustments to, for example, frequency allocation tables (FAT)/number of channels, threshold/comfort levels, etc.

Certain adjustments can be made to minimize cycle usage for the parallel processing path. For example, complex noise reduction strategies can be disabled. Adjustments can be made to ensure environmental awareness in all circumstances. For example, the shared audio can always have no/only basic noise reduction applied, and always use omnidirectional processing. Additionally, delays can be added to the shared or same-side audio to synchronize the final outputs for the recipient. For example, if it is known one data link has higher latency (e.g., 2.4 GHz) a delay can be added to the other data link to synchronize the data over the data links.

In some embodiments, sound processing unit 106R can send different types of data to implantable component 112R and implantable component 112L. For example, sound processing unit 106R can transmit stimulation data to implantable component 112R and can transmit audio data to implantable component 112L. In this example, sound processing unit 106R can receive sound signals, perform the processing steps 702R, 704R, 706R, and 708R, and transmit stimulation data to implantable component 112R. Sound processing unit 106R can additionally perform directional processing of the sound signals at 702L and then transmit the processed sound signals to implantable component 112L without performing additional processing. In some embodiments, sound processing unit 106R can send unprocessed audio signals to implantable component 112L. Implantable component 112L can process the unprocessed or partially processed audio signal and output stimulation data to the recipient.

Different types of data can be transmitted to ipsilateral implantable components and contralateral implantable components for different reasons and based on different factors. For example, the types of links, the generation/processing capabilities of the contralateral implantable components, limitations on processing power available on the available sound processor, and additional factors can contribute to a type of data transmitted to each implantable component.

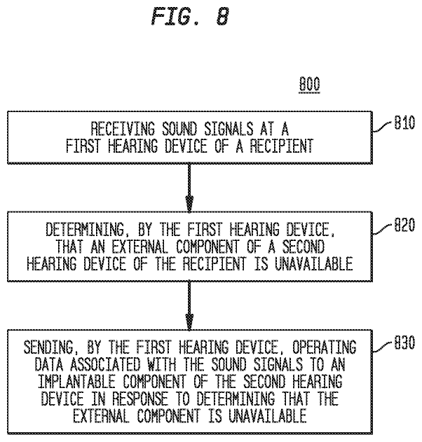

FIG. 8 is a flow chart illustrating a method 800 for performing parallel signal processing when a sound processing unit of a hearing device in a bilateral hearing device system is unavailable. The method can be performed by a hearing device, such as a sound processing unit 106L/106R, a hearing aid 150, totally implantable cochlear implant 612, or another device.

At 810, sound signals are received at a first hearing device at a recipient. The hearing device can be configured to deliver treatment to a first ear of the recipient. For example, a microphone or sound input device of the hearing device can receive sound signals for delivering stimulation data to the first ear of the recipient.

At 820, the first hearing device can determine that an external component of a second hearing device of the recipient is unavailable. The second hearing device can be configured to deliver treatment, such as electrical stimulation data, to a second ear of the recipient. The first hearing device can determine that a sound processing unit of a contralateral hearing device is unavailable. In one example, the first hearing device can determine that the second hearing device is unavailable based on a link between the first hearing device and the hearing device being unavailable. In another example, the first hearing device can determine that the sound processing unit of the second hearing device is unavailable based on receiving a message from an implantable component of the contralateral hearing device.

At 830, the first hearing device transmits operating data associated with the sound signals to an implantable component of the second hearing device in response to determining that the external component is unavailable. In one example, the operating data can include stimulation data. In another example, the operating data includes unprocessed audio data or at least partially processed audio data. In some embodiments, a type of the operational data can be based on a type of link established between the first hearing device and the implantable component of the second hearing device.

By sending the operating data to the second hearing device, the recipient of the first and second hearing devices can continue to receive signals in both ears when one of the hearing devices is unavailable.

Certain embodiments have been described herein with reference to arrangements in which the two devices performing the parallel processor are physically separated. However, it is to be appreciated that, in certain embodiments, the two devices can be part of a same physical structure, yet still operate as two separate devices. In one such example, first and second hearing devices can be integrated into a single unit, such as in a pair of glass/spectacles (e.g., sending mic signals on left and right sides to respective implants). In this example, there could be a failure at least of one of the microphones, processors, etc., which could be addressed using the techniques described elsewhere herein.

Merely for ease of description, the techniques presented herein have primarily described herein with reference to an illustrative medical device system, namely a cochlear implant system that delivers electrical stimulation to both ears of a recipient. However, it is to be appreciated that the techniques presented herein can also be used with a variety of other medical devices that, while providing a wide range of therapeutic benefits to recipients, patients, or other users, can benefit from the techniques presented. For example, a cochlear implant system in accordance with embodiments presented herein can also deliver acoustic stimulation to one or both ears of the recipient (e.g., one or more of the cochlear implants is an electro-acoustic cochlear implant). It is also to be appreciated that the two cochlear implants of a cochlear implant system in accordance with embodiments presented need not be identical with respect to, for example, the number of electrodes used to electrically stimulate the cochlea, the type of stimulation delivered, etc.

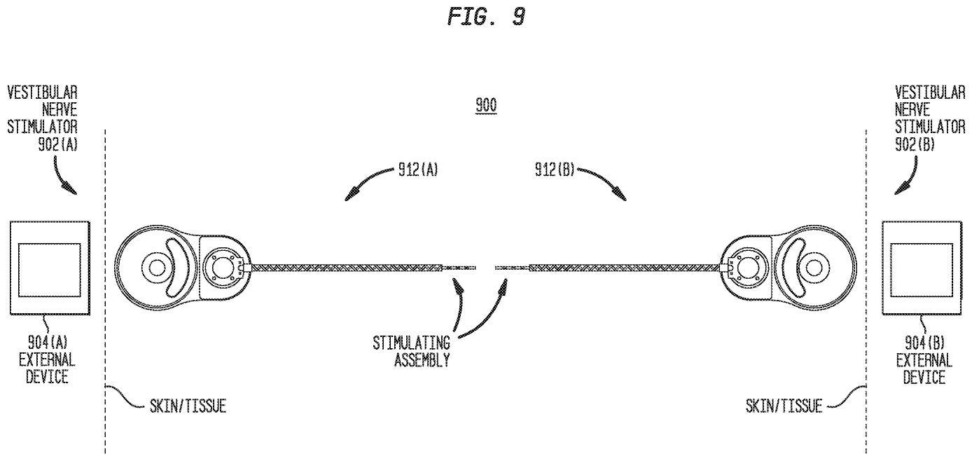

Furthermore, it is to be appreciated that the techniques presented herein can be used with other systems including two or more devices, such as systems including one or more personal sound amplification products (PSAPs), one or more acoustic hearing aids, one or more bone conduction devices, one or more middle ear auditory prostheses, one or more direct acoustic stimulators, one or more other electrically simulating auditory prostheses (e.g., auditory brain stimulators), one or more vestibular devices (e.g., vestibular implants), one or more visual devices (i.e., bionic eyes), one or more sensors, one or more pacemakers, one or more drug delivery systems, one or more defibrillators, one or more functional electrical stimulation devices, one or more catheters, one or more seizure devices (e.g., devices for monitoring and/or treating epileptic events), one or more sleep apnea devices, one or more electroporation devices, one or more remote microphone devices, one or more consumer electronic devices, etc. For example, FIGS. 9, 10, and 11 are schematic diagrams of alternative systems that can implement aspects of the techniques presented herein.

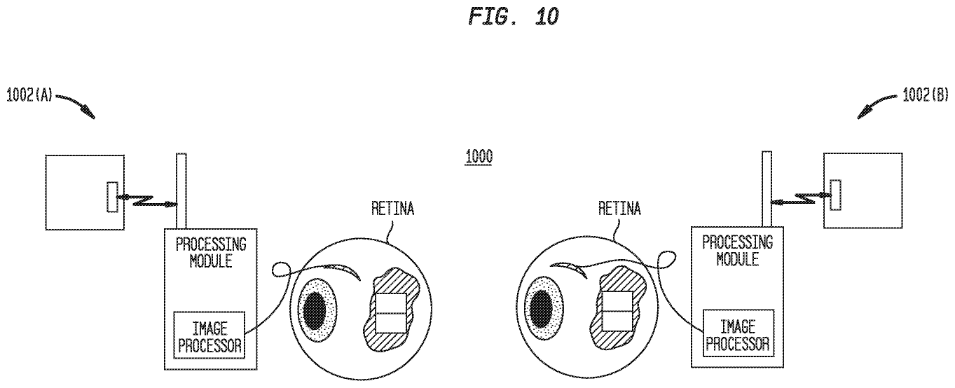

More specifically, FIG. 9 is a schematic diagram illustrating an example vestibular system 900 that can be configured to perform synchronized spectral analysis, in accordance with certain embodiments presented herein. In this example, the vestibular system 900 comprises a first vestibular stimulator 902(A) and a second vestibular stimulator 902(B). The first vestibular stimulator 902(A) comprises an external device 904(A) and an implantable component 912(A), while the second vestibular stimulator 902(B) comprises an external device 904(B) and an implantable component 912(B). In accordance with certain embodiments presented herein, the first vestibular stimulator 902(A) (e.g., external device 904(A) and/or implantable component 912(A)) and/or the second vestibular stimulator 902(B) (e.g., external device 904(B) and/or implantable component 912(B)) are configured to implement aspects of the techniques presented herein to perform synchronized spectral analysis of received/input signals (e.g., audio signals, sensor signals, etc.). In general, the external device of a vestibular system (e.g., external devices 904(A) and/or 904(B)) can perform analysis using movement sensors in each respective external device and, as such, the operating data sent between devices (as described above) can include spatial information. In addition, the vestibular stimulator(s) 902(A) and/or 902(B) can, in different embodiments, generate an electric, mechanical, and/or optical output FIG. 10 is a schematic diagram illustrating an example retinal prosthesis system 1000 that can be configured to perform synchronized spectral analysis, in accordance with certain embodiments presented herein. In this example, the retinal prosthesis system 1000 comprises a first retinal prosthesis 1002(A) and a second retinal prosthesis 1002(B). In accordance with certain embodiments presented herein, the first retinal prosthesis 1002(A) and/or the second retinal prosthesis 1002(B) are configured to implement aspects of the techniques presented herein to perform synchronized spectral analysis of received/input signals (e.g., light signals, sensor signals, etc.).

As previously described, the technology disclosed herein can be applied in any of a variety of circumstances and with a variety of different devices. While the above-noted disclosure has been described with reference to medical device, the technology disclosed herein can be applied to other electronic devices that are not medical devices. For example, this technology can be applied to, e.g., ankle or wrist bracelets connected to a home detention electronic monitoring system, or any other chargeable electronic device worn by a user.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments can be combined with another in any of a number of different manners.

1. A method comprising:
receiving ambient signals at a first device of a recipient;
determining, by the first device, that an external component of a second device of the recipient is unavailable; and
sending from the first device, in response to determining that the external component of the second device is unavailable, operating data associated with the ambient signals to an implantable component of the second device.

2. The method of claim 1, wherein the operating data includes stimulation data.

3. The method of claim 1, wherein the operating data includes at least partially processed audio data.

4. The method of claim 1, wherein the first device and the second device communicate via a wireless link and wherein a type of the operating data that is transmitted to the implantable component of the second device is based on a type of the wireless link.

5. The method of claim 1, further comprising:
processing, by the first device, the ambient signals in a first manner for output to the recipient via the first device; and
processing, by the first device, the ambient signals in a second manner to create the operating data for transmission to the second device.

6. The method of claim 5, wherein processing the ambient signals in the second manner includes:
receiving, from the second device, parameters for processing the ambient signals; and
processing the ambient signals in the second manner based on the parameters.

7. The method of claim 5, wherein processing the sound signals ambient signals in the second manner includes:
processing the ambient signals based on characteristics of hardware associated with the first hearing device.

8. The method of claim 5, further comprising:
adding a delay when processing the ambient signals in the first manner or the second manner.

9. The method of claim 1, wherein the ambient signals are sound signals, and wherein the first device and the second device are hearing devices.

10. (canceled)

11. (canceled)

12. (canceled)

13. An implantable medical device system, comprising:
a first medical device configured to deliver treatment to a first portion of a recipient, wherein the first medical device comprises an external component and an implantable component; and
a second medical device configured to deliver treatment to a second portion of a recipient, wherein the second medical device is configured to determine that the external component of the first medical device is unavailable and, in response to determining that the external component the first medical device is unavailable, send operating data to the implantable component.

14. (canceled)

15. The implantable medical device system of claim 13, wherein the operating data includes at least one stimulation data or at least partially processed audio data.

16. (canceled)

17. The implantable medical device system of claim 13, wherein the operating data includes at least one of unprocessed audio data or channelized audio data.

18. (canceled)

19. The implantable medical device system of claim 13, wherein the first medical device and the second medical device communicate via a wireless link and wherein a type of the operating data that is sent to the implantable component of the second medical device is based on a type of the wireless link.

20. The implantable medical device system of claim 13, wherein the second medical device is further configured to process the operating data in a first manner for delivering the treatment to the first portion of the recipient and process the operating data in a second manner for delivering the treatment to the second portion of the recipient.

21. The implantable medical device system of claim 20, wherein, when processing the operating data in the second manner, the second medical device is further configured to:
receive, from the first medical device, parameters for processing the operating data; and
process the operating data in the first manner based on the parameters.

22. (canceled)

23. (canceled)

24. The implantable medical device system of claim 13, wherein the operating data is sent to the implantable component in response to an input from the recipient.

25. (canceled)

26. (canceled)

27. One or more non-transitory computer readable storage media comprising instructions that, when executed by a processor of a first device of a recipient, cause the processor to:
receive environmental signals;
determine that an external component of a second device of the recipient is unavailable; and
transmit operating data associated with the environmental signals to an implantable component of the second device in response to determining that the external component is unavailable.

28. The one or more non-transitory computer readable storage media of claim 27, wherein the operating data includes at least one of stimulation data or at least partially processed audio data.

29. (canceled)

30. The one or more non-transitory computer readable storage media of claim 27, wherein the instructions further cause the processor to communicate with the first hearing device via a wireless link, and wherein a type of the operating data that is transmitted to the implantable component of the second hearing device is based on a type of the wireless link.

31. The one or more non-transitory computer readable storage media of claim 27, wherein the instructions further cause the processor to:
process the environmental signals in a first manner for output to the recipient via the first device; and
process the environmental signals in a second manner to create the operating data for transmission to the second hearing device.

32-42. (canceled)
